(19) United States
(12) Patent Application Publication
(10) Pub. No.: US 2024/0290446 A1
(43) Pub. Date: Aug. 29, 2024

(54) PATIENT INFORMATION PROCESSING METHOD, PATIENT INFORMATION PROCESSING DEVICE, AND PATIENT INFORMATION PROCESSING SYSTEM

(71) Applicants: ST. MARIANNA UNIVERSITY SCHOOL OF MEDICINE, Kawasaki-shi (JP); NIHON KOHDEN CORPORATION, Tokyo (JP)

(72) Inventors: Shigeki FUJITANI, Kawasaki-shi (JP); Toru Yoshida, Kawasaki-shi (JP); Itaru Fukuhara, Tokorozawa-shi (JP); Mitsuhiro Oura, Tokorozawa-shi (JP); Syuhei Kawamura, Tokorozawa-shi (JP)

(73) Assignees: ST. MARIANNA UNIVERSITY SCHOOL OF MEDICINE, Kawasaki-shi (JP); NIHON KOHDEN COPRPRATION, Tokyo (CA)

(21) Appl. No.: 18/587,199

(22) Filed: Feb. 26, 2024

(30) Foreign Application Priority Data
Feb. 27, 2023 (JP) 2023-028468

(51) Int. Cl. G16H 10/60; G16H 50/30

(52) U.S. Cl. CPC G16H 10/60; G16H 50/30

(57) ABSTRACT

The patient information processing method includes a step of obtaining a plurality of pieces of vital data of a patient, a step of obtaining information indicating a content and a time of a treatment performed on the patient, and a step of causing a display to display information indicating at least one of the plurality of pieces of vital data or an index associated with the plurality of pieces of vital data, and to display information indicating the content and the time of the treatment.

### CROSS-REFERENCE TO RELATED APPLICATIONS

This application is based on and claims priority under 35 USC 119 from Japanese Patent Application No. 2023-028468 filed on Feb. 27, 2023, the entire content of which is incorporated herein by reference.

### TECHNICAL FIELD

The present disclosure relates to a patient information processing method, a patient information processing device, and a patient information processing system. Furthermore, the present disclosure relates to a program that causes a computer to execute the patient information processing method, and a non-transitory computer readable storage medium storing the program.

### BACKGROUND ART

In the world, a pandemic due to a novel coronavirus infectious disease (COVID-19) still rages, and in a specific medical institution, the management of a severe patient has become a very important factor with an explosive increase of a patient infected with the novel coronavirus. In particular, in a medical field, vital data of a patient and a vital score indicating a condition of a patient (for example, National Early Warning Score (NEWS)) are effectively used for the management of a severe patient.

JP2019-170851A discloses a patient information processing system configured to calculate a vital score for each patient, based on vital data of the patient, and configured to display a patient information list screen including a plurality of pieces of patient information. A medical worker can grasp the condition of each patient by recognizing the vital score and the vital data of each patient displayed on the patient information list screen.

In order to more comprehensively determine the condition of a severe patient who is treated in an intensive care unit, it is preferable that a determination index different from vital data, vital scores, body findings, image data, and the like of the patient can also be used. For example, by displaying information indicating a degree of intervention of a medical worker with respect to the patient on the patient information list screen, the medical worker can comprehensively determine the condition of the patient. In this manner, there is still room for consideration of a medical tool for supporting the medical worker to comprehensively determine the condition of the patient.

### SUMMARY OF INVENTION

A first aspect of non-limiting embodiments of the present disclosure relates to provide a patient information processing method, a patient information processing device, and a patient information processing system for supporting a medical worker to comprehensively determine a condition of a patient. A second aspect of non-limiting embodiments of the present disclosure relates to provide a program that causes a computer to execute the patient information processing method, and a non-transitory computer readable storage medium storing the program.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to a first aspect of the present disclosure, there is provided a patient information processing method executed by one or more processors, the patient information processing method including:
a step of obtaining a plurality of pieces of vital data of a patient;
a step of obtaining information indicating a content and a time of a treatment performed on the patient; and
a step of causing a display to display information indicating at least one of the plurality of pieces of vital data or an index associated with the plurality of pieces of vital data, and to display information indicating the content and the time of the treatment.

According to a second aspect of the present disclosure, there is provided a patient information processing method executed by one or more processors, the patient information processing method including:
a step of obtaining a plurality of pieces of vital data of a patient;
a step of calculating intervention information indicating a degree of intervention of a medical worker with respect to the patient, based on at least one of information related to a treatment performed on the patient or information indicating a hospitalization time of the patient; and
a step of causing a display to display the intervention information and information indicating at least one of the plurality of pieces of vital data or an index associated with the plurality of pieces of vital data.

According to a third aspect of the present disclosure, there is provided a patient information processing device including:
one or more processors; and
one or more memories configured to store a computer-readable instruction,
in which, in a case where the computer-readable instruction is executed by the one or more processors, the patient information processing device is configured to:
   obtain a plurality of pieces of vital data of a patient;
   obtain information indicating a content and a time of a treatment performed on the patient; and
   output information indicating at least one of the plurality of pieces of vital data or an index associated with the plurality of pieces of vital data, and information indicating the content and the time of the treatment.

According to a fourth aspect of the present disclosure, there is provided a patient information processing device including:
one or more processors; and
one or more memories configured to store a computer-readable instruction,
in which, in a case where the computer-readable instruction is executed by the one or more processors, the patient information processing device is configured to:
   obtain a plurality of pieces of vital data of a patient;
   calculate intervention information indicating a degree of intervention of a medical worker with respect to the patient, based on at least one of information related to a treatment performed on the patient or information indicating a hospitalization time of the patient; and
   output the intervention information and information indicating at least one of the plurality of pieces of vital data or an index associated with the plurality of pieces of vital data.

According to a fifth aspect of the present disclosure, there is provided a patient information processing system including:
a server; and
a display terminal communicably connected to the server,
in which the patient information processing system is configured to:
   obtain a plurality of pieces of vital data of a patient;
   obtain information indicating a content and a time of a treatment performed on the patient; and
   cause a display to display information indicating at least one of the plurality of pieces of vital data or an index associated with the plurality of pieces of vital data, and information indicating the content and the time of the treatment.

According to a sixth aspect of the present disclosure, there is provided a patient information processing system including:
a server; and
a display terminal communicably connected to the server,
in which the patient information processing system is configured to:
   obtain a plurality of pieces of vital data of a patient;
   calculate intervention information indicating a degree of intervention of a medical worker with respect to the patient, based on at least one of information related to a treatment performed on the patient or information indicating a hospitalization time of the patient; and
   cause a display to display the intervention information and information indicating at least one of the plurality of pieces of vital data or an index associated with the plurality of pieces of vital data.

### BRIEF DESCRIPTION OF DRAWINGS

Exemplary embodiment(s) of the present invention will be described in detail based on the following figures, wherein:

FIG. 1 is a diagram illustrating an example of a configuration of a patient information processing system according to an embodiment of the present disclosure (hereinafter, referred to as the present embodiment).

FIG. 2 is a diagram illustrating an example of a hardware configuration of a server.

FIG. 3 is a diagram illustrating an example of various tables included in an in-hospital information database.

FIG. 4 is a diagram illustrating an example of a patient information table.

FIG. 5 is a diagram illustrating an example of a method of calculating a NEWS score.

FIG. 6 is a diagram illustrating an example of a hardware configuration of a display terminal.

FIG. 7 is a sequence diagram illustrating a patient information processing method according to a first embodiment of the present disclosure.

FIG. 8 is a diagram illustrating an example of a patient information list screen according to the first embodiment.

FIG. 9 is a diagram illustrating an example of a patient information list screen according to a modification of the first embodiment.

FIG. 10 is a sequence diagram illustrating a patient information processing method according to a second embodiment of the present disclosure.

FIG. 11 is a diagram illustrating an example of a patient information table according to the second embodiment.

FIG. 12 is a diagram illustrating an example of a patient information list screen according to the second embodiment.

FIG. 13 is a sequence diagram illustrating a patient information processing method according to a third embodiment of the present disclosure.

FIG. 14 is a diagram illustrating an example of a patient information table according to the third embodiment.

FIG. 15 is a diagram illustrating an example of a patient information list screen according to the third embodiment.

### DESCRIPTION OF EMBODIMENTS

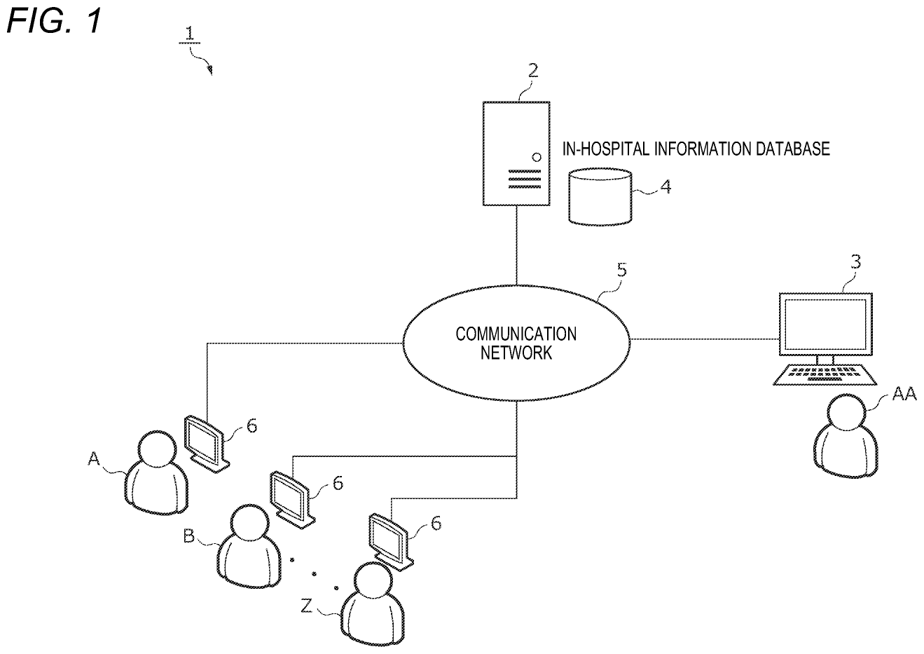

Hereinafter, the present embodiment will be described with reference to the drawings First, a patient information processing system 1 according to the present embodiment will be described below with reference to FIG. 1. FIG. 1 is a diagram illustrating an example of a configuration of the patient information processing system 1 (hereinafter, referred to as a processing system 1) according to the present embodiment. As illustrated in FIG. 1, the processing system 1 can include a server 2, a plurality of bed-side monitors 6, and a display terminal 3. The server 2 is communicably connected to the plurality of bed-side monitors 6 and the display terminal 3 via a communication network 5.

The communication network 5 may be an in-hospital network including, for example, a local area network (LAN) or a wide area network (WAN). In addition, the communication network 5 may include an in-hospital network and the Internet. In this case, the server 2 may be a server installed in the in-hospital network or an external server (for example, a cloud server) installed outside the in-hospital network.

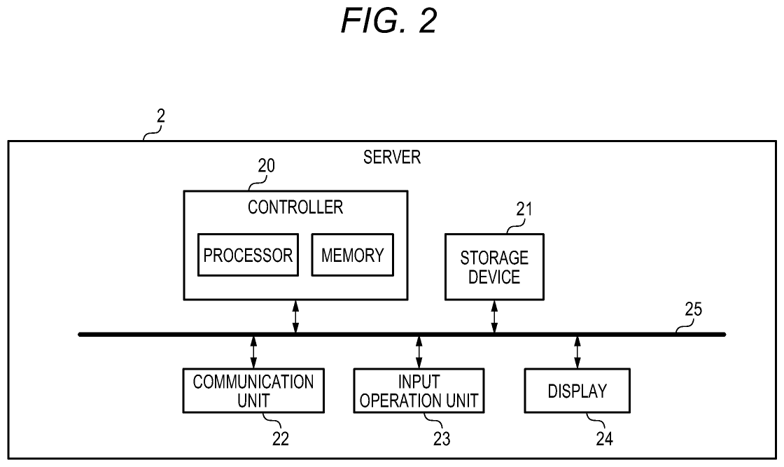

The server 2 can include an in-hospital information database 4. The in-hospital information database 4 is built in, for example, a storage device 21 (see FIG. 2). The in-hospital information database 4 may be built in another database server communicably connected to the server 2.

Next, a hardware configuration of the server 2 will be described with reference to FIG. 2. FIG. 2 is a diagram illustrating an example of the hardware configuration of the server 2.

As illustrated in FIG. 2, the server 2 can include a controller 20, the storage device 21, a communication unit 22, an input operation unit 23, and a display 24. These components are communicably connected to one another via a bus 25.

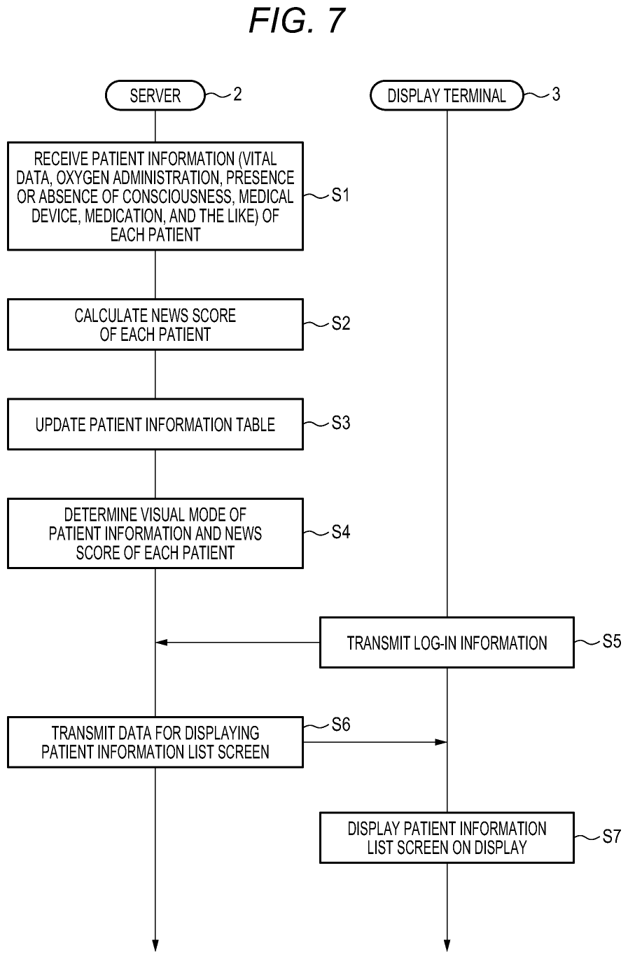

The controller 20 may include one or more memories and one or more processors. Each memory is configured to store a computer-readable instruction (program). For example, the memory may include a read only memory (ROM) in which various programs are stored, and a random access memory (RAM) having a plurality of work areas in which various programs executed by the one or more processors are stored. Each processor may include at least one of, for example, a central processing unit (CPU), a micro processing unit (MPU), and a graphics processing unit (GPU). The CPU may include a plurality of CPU cores. The GPU may include a plurality of GPU cores. The processor may be configured to load a program designated from various programs incorporated in the storage device 21 or ROM into RAM, and configured to execute various processes in cooperation with the RAM. In particular, the processor loads a patient information processing program that executes a series of processes (for example, see FIG. 7) of the server 2 into the RAM, and executes the program in cooperation with the RAM, whereby the controller 20 executes the series of processes illustrated in FIG. 7. Details of the patient information processing program will be described later.

The storage device 21 is a storage device (storage) such as a hard disk drive (HDD), a solid state drive (SSD), or a flash memory, and is configured to store programs and various data. The patient information processing program may be incorporated in the storage device 21. In addition, the in-hospital information database 4 may be stored in the storage device 21.

The communication unit 22 is configured to connect the server 2 to the communication network 5. Specifically, the communication unit 22 may include various wired connection terminals for communicating with each device disposed on the communication network 5. In addition, the communication unit 22 may include a wireless communication module for wirelessly communicating with each device. The input operation unit 23 is, for example, a touch panel, a mouse, and/or a keyboard, which are overlaid and disposed over the display 24. The input operation unit 23 is configured to receive an input operation of an operator and configured to generate an operation signal corresponding to the input operation of the operator. The display 24 is configured by, for example, a liquid crystal panel or an organic EL panel.

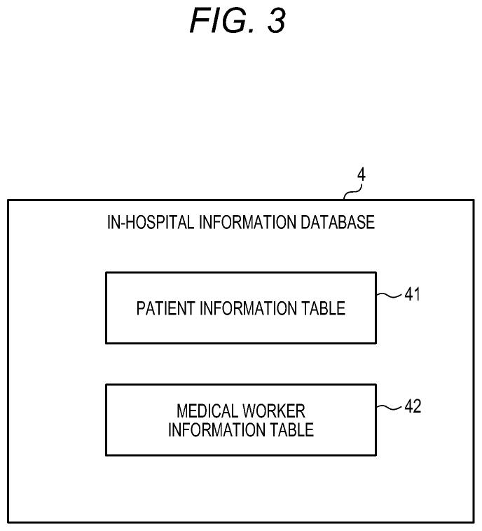

Next, the in-hospital information database 4 built in the storage device 21 will be described with reference to FIG. 3. As illustrated in FIG. 3, the in-hospital information database 4 can include a patient information table 41 and a medical worker information table 42.

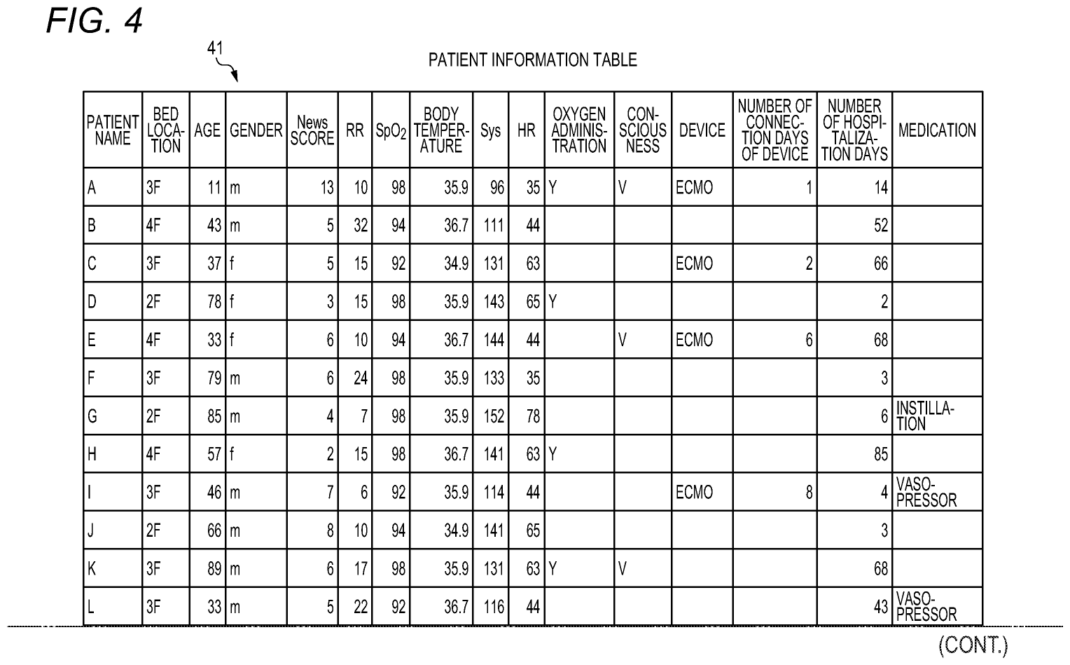

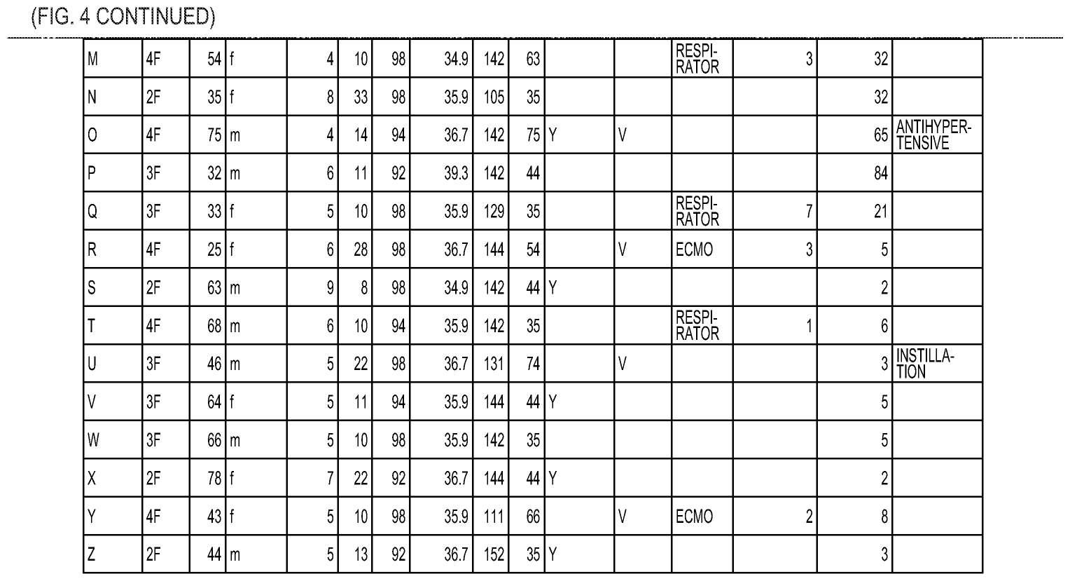

FIG. 4 is a diagram illustrating an example of the patient information table 41. As illustrated in FIG. 4, the patient information table 41 can include attribute information of a patient, information indicating a NEWS score of the patient, information indicating vital data of the patient, information indicating presence or absence of oxygen administration, and information indicating presence or absence of consciousness of the patient. Furthermore, the patient information table 41 can include information related to a medical device connected to the patient, information indicating the number of hospitalization days of the patient, and information indicating medication given to the patient. The information included in the patient information table 41 may be updated at predetermined time intervals.

The attribute information of the patient can include information indicating a patient name, a bed location, an age, and a gender. The information indicating the vital data of the patient can include information indicating a respiration rate (RR), an oxygen saturation (SpO2), a body temperature, a systolic blood pressure (Sys), and a heart rate (HR).

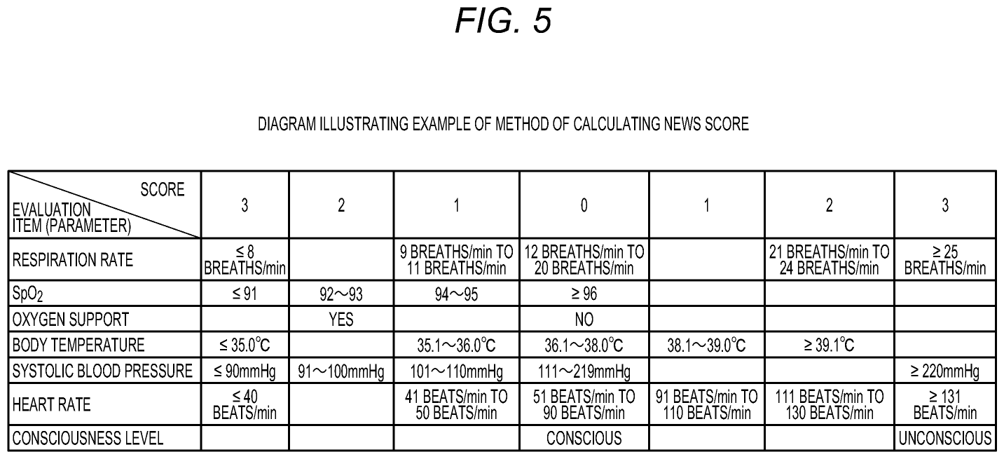

In the description of the present embodiment, the NEWS score is calculated as an example of the vital score indicating the condition of the patient. For example, as illustrated in FIG. 5, the server 2 is configured to calculate the NEWS score, based on each piece of vital data, the presence or absence of oxygen administration, and the presence or absence of consciousness of the patient. Specifically, the server 2 is configured to calculate a sub score of each of the plurality of pieces of vital data, in accordance with a comparison between each of the plurality of pieces of vital data and a reference range set for each of the plurality of pieces of vital data.

For example, in a case where the respiration rate (RR) is 12 breaths/min to 20 breaths/min, the sub score related to the respiration rate is zero points. In a case where the respiration rate is nine breaths/min to 11 breaths/min, the sub score related to the respiration rate is one point. In a case where the respiration rate is 21 breaths/min to 24 breaths/min, the sub score related to the respiration rate is two points. In a case where the respiration rate is eight breaths/min or less or 25 breaths/min or more, the sub score related to the respiration rate is three points.

Furthermore, the server 2 is configured to calculate a sub score related to oxygen administration and a sub score related to consciousness of the patient. For example, in a case where oxygen administration is not performed, the sub score related to oxygen administration is two points. On the other hand, in a case where oxygen administration is not performed, the sub score related to oxygen administration is zero points. In addition, in a case where the patient is unconscious, the sub score related to consciousness of the patient is three points. On the other hand, in a case where the patient is conscious, the sub score related to consciousness of the patient is zero points.

In this manner, the server 2 is configured to calculate the NEWS score of the patient by summing up the calculated sub scores. For example, as illustrated in FIG. 4, the respiration rate (RR) of a patient A is ten beats/min, and therefore, the sub score related to the respiration rate is one point. The oxygen saturation (SpO2) of the patient A is 98%, and therefore, the sub score related to the oxygen saturation is zero points. The body temperature of the patient A is 35.9° C., and therefore, the sub score related to the body temperature is one point. The systolic blood pressure (Sys) of the patient A is 96 mmHg, and therefore, the sub score related to the systolic blood pressure is two points. The heart rate (HR) of the patient A is 35 beats/min, and therefore, the sub score related to the heart rate is three points. Oxygen administration is performed on the patient A, and therefore, the sub score related to oxygen administration is three points. The patient A is unconscious, and therefore, the sub score related to consciousness of the patient is three points. In this manner, the NEWS score of the patient A is 13 points by summing up the sub scores (=1+0+1+2+3+3+3).

In the present embodiment, the vital score (example of an index associated with the plurality of pieces of vital data) is not limited to the NEWS score. For example, as another example of the vital score, SOFA, qSOFA, APACHE2, BSAS, NIHSS, or the like may be adopted. In addition, in the present embodiment, the respiration rate, the oxygen saturation, the body temperature, the systolic blood pressure, and the heart rate are obtained as the vital data of the patient in order to calculate the NEWS score, but the type of the vital data of the patient may be changed in accordance with the type of the vital score to be adopted.

In addition, each piece of vital data included in the patient information table 41 is updated at predetermined time intervals. As illustrated in FIG. 1, the vital data of each patient is transmitted from the plurality of bed-side monitors 6 (example of a patient monitor) to the server 2, via the communication network 5, at predetermined time intervals. Each of the plurality of bed-side monitors 6 is connected to one of patients A to Z. Each of the bed-side monitors 6 includes a plurality of vital sensors for measuring vital data such as a respiration rate and a heart rate of a patient. The plurality of vital sensors may include, for example, a respiration sensor, an SpO2 sensor, a body temperature sensor, a blood pressure sensor, and an electrocardiogram sensor.

The information related to a medical device connected to the patient can include information indicating a type of the medical device and information indicating the number of connection dates of the medical device. In a case where an extracorporeal membrane oxygenation (ECMO) is connected to the patient, information indicating ECMO is input as the information indicating the type of the medical device connected to the patient. In a case where a respirator is connected to the patient, information indicating the respirator is input as the information indicating the type of the medical device connected to the patient. The medical device connected to the patient is not limited to the ECMO or the respirator, and may be a medical device such as a CO2 sensor or a pacemaker. In addition, in a case where the number of days of the ECMO connected to the patient is 14 days, 14 days is input as the information indicating the number of connection days of the medical device. In the present embodiment, the information indicating the type of the medical device is an example of information indicating a content of a treatment performed on the patient. The information indicating the number of connection days of the medical device is an example of information indicating a time of the treatment performed on the patient.

The information indicating the number of hospitalization days (hospitalization time) of the patient may be calculated by the server 2, based on a difference between a hospitalization date of the patient and a current date. In this manner, the information indicating the number of hospitalization days of the patient is updated every day. The number of hospitalization days of the patient may be expressed in a format including an hour and minute unit. For example, in a case where the patient A is hospitalized at 5:00 on Apr. 1, 2022, the number of hospitalization days of the patient A at 17:30 on Apr. 1, 2022 may be output as 0 days, 12 hours, and 30 minutes.

The information indicating medication given to the patient includes information indicating a type of medication. For example, in a case where a vasopressor is administered to a patient, the vasopressor is input as the information indicating medication. In addition, the information indicating medication may include information indicating the number of times or the days of medication, in addition to the information indicating the type of medication. In the present embodiment, the information indicating the type of the medication is an example of the information indicating the content of the treatment performed on the patient. The information indicating the number of times or the days of medication is an example of the information indicating the time of the treatment performed on the patient.

Each of the information indicating presence or absence of oxygen administration, the information indicating presence or absence of consciousness of the patient, the information related to the medical device, the information indicating the hospitalization date of the patient, and the information related to medication may be directly input to the server 2 through the input operation unit 23 of the server 2. Alternatively, the server 2 may be configured to receive the information from an electronic medical record server (not illustrated) disposed on the communication network 5 at predetermined time intervals.

In addition, the medical worker information table 42 illustrated in FIG. 3 can include information indicating attributes of each medical worker (name, department to which a medical worker belongs, job type, years of experience, and the like).

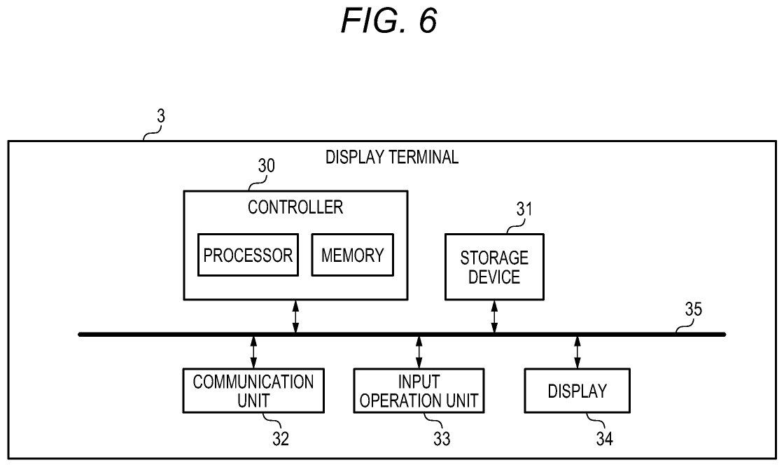

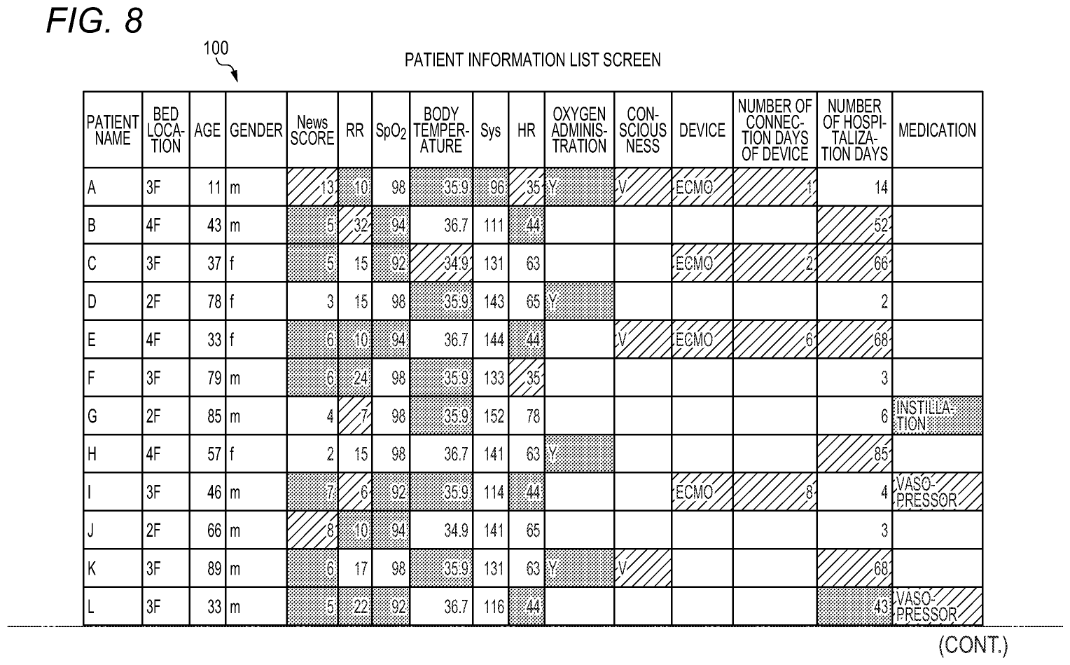

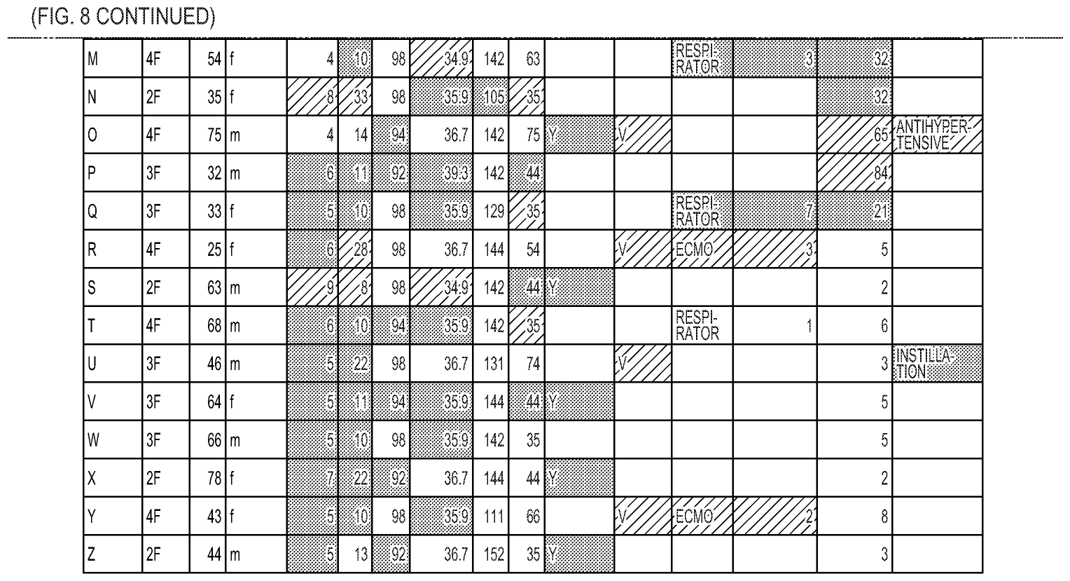

Next, a hardware configuration of the display terminal 3 will be described below with reference to FIG. 6. FIG. 6 is a diagram illustrating an example of the hardware configuration of the display terminal 3. As illustrated in FIG. 6, the display terminal can 3 include a controller 30, a storage device 31, a communication unit 32, an input operation unit 33, and a display 34. These components are communicably connected to one another via a bus 35. A type of the display terminal 3 is not particularly limited as long as the display terminal 3 is a display terminal managed by a medical worker and has a function (for example, a WEB browser) capable of displaying a patient information list screen (see FIG. 8 and the like) to be described later. The display terminal 3 may be a patient monitor, a personal computer, a workstation, a smartphone, a tablet, or a wearable device (for example, an AR glass or a head-mounted display) worn on a body (for example, an arm or a head) of a medical worker.

The controller 30 can include one or more memories and one or more processors. Each memory is configured to store a computer-readable instruction (program). For example, the memory can include a ROM and a RAM. Each processor can include at least one of a CPU, an MPU, and a GPU, for example. The processor may be configured to load a program designated from various programs incorporated in the storage device 31 or ROM into RAM, and configured to execute various processes in cooperation with the RAM. The storage device 31 is a storage device such as an HDD, an SSD, or a flash memory, and is configured to store programs and various data.

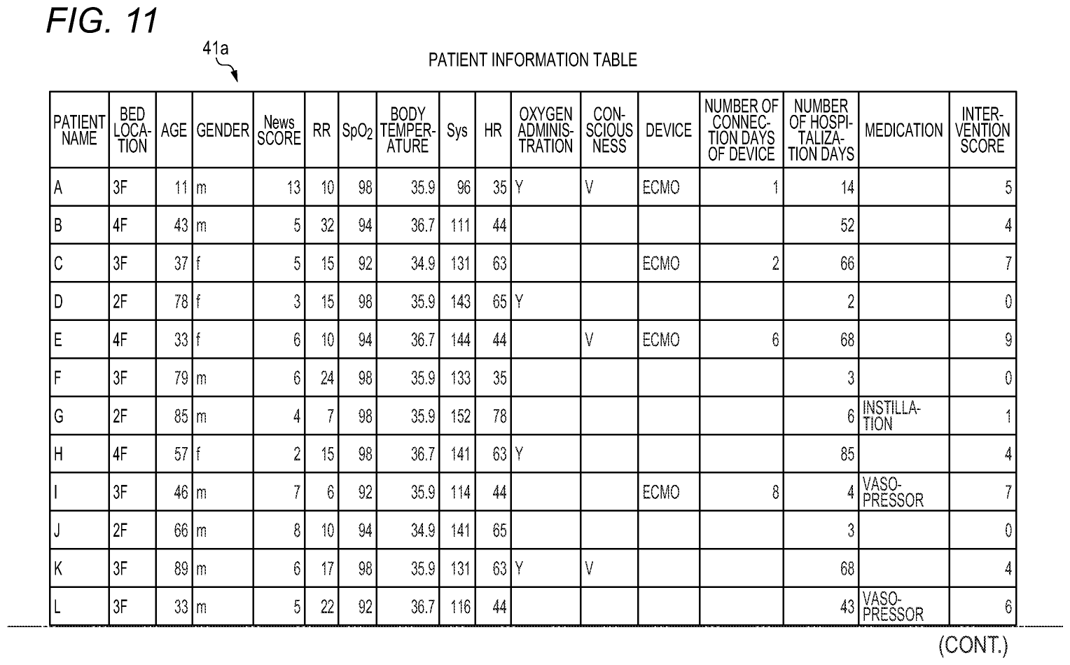

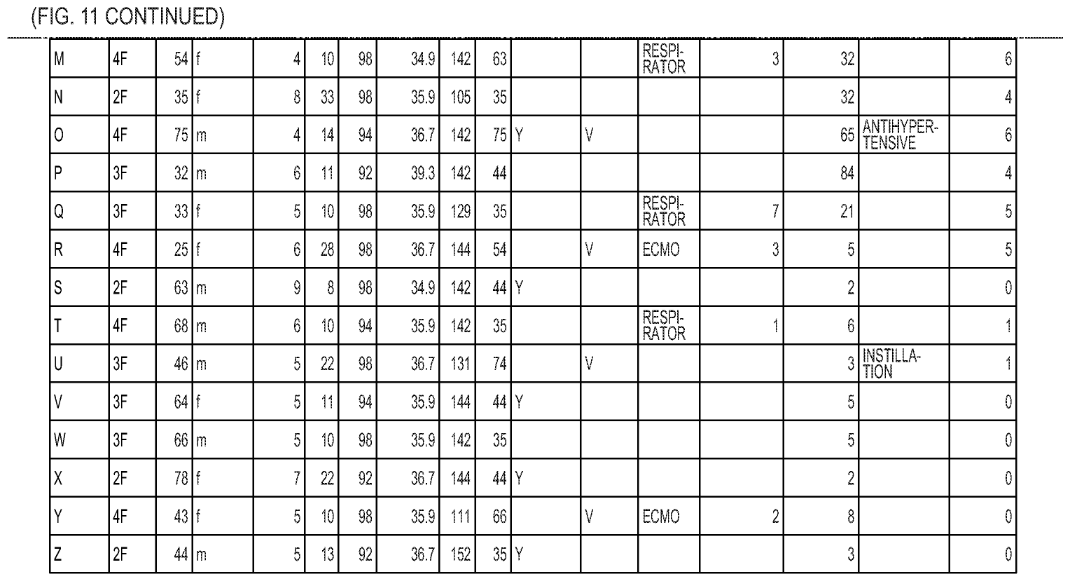

The communication unit 32 is configured to connect the display terminal 3 to the communication network 5. Specifically, the communication unit 32 may include various wired connection terminals for communicating with each device disposed on the communication network 5. In addition, the communication unit 32 may include a wireless communication module for wirelessly communicating with each device. The input operation unit 33 is, for example, a touch panel, a mouse, and/or a keyboard, which are overlaid and disposed over the display 34. For example, according to an input operation by a medical worker AA on the input operation unit 33, an input operation signal indicating the input operation by the medical worker AA is transmitted from the input operation unit 33 to the controller 30. Thereafter, the controller 30 executes a predetermined process according to the received input operation signal. The display 34 is configured by, for example, a liquid crystal panel or an organic EL panel. A patient information list screen (see FIG. 11) to be described later is displayed on a display screen of the display 34. In a case where the display terminal 3 is a head-mounted display, the patient information list screen may be disposed in a virtual space (Metaverse).

### First Embodiment

Next, a patient information processing method according to a first embodiment of the present disclosure will be described below with reference to FIGS. 7 and 8. FIG. 7 is a sequence diagram illustrating the patient information processing method according to the first embodiment. FIG. 8 is a diagram illustrating a patient information list screen 100 according to the first embodiment. As illustrated in FIG. 7, the patient information processing method according to the first embodiment is executed by cooperation between the server 2 and the display terminal 3. In addition, in the following description, it is assumed that the medical worker AA accesses the server 2 through the display terminal 3.

In step S1, the server 2 receives patient information of each of the patients A to Z. In this respect, the server 2 receives a plurality of pieces of vital data (respiration rate, oxygen saturation, body temperature, systolic blood pressure, and heart rate) of each of the patients A to Z, from the plurality of bed-side monitors 6 connected to the patients A to Z via the communication network 5. As described above, the server 2 may obtain the vital data of the patients A to Z from the bed-side monitors 6, via the communication network 5, at predetermined time intervals (for example, every day). Furthermore, the server 2 obtains, as the patient information of each of the patients A to Z, information indicating presence or absence of oxygen administration, information indicating presence or absence of consciousness of the patient, information related to a medical device connected to the patient, information indicating a hospitalization date of the patient, and information related to medication. As described above, the information may be directly input to the server 2 through the input operation unit 23 of the server 2, or may be received from an electronic medical record server disposed on the communication network 5.

Next, in step S2, the server 2 calculates a NEWS score of each of the patients A to Z based on the plurality of pieces of vital data of each of the patients A to Z, presence or absence of oxygen administration of each of the patients A to Z, and presence or absence of consciousness of each of the patients A to Z. As described above, the server 2 is configured to calculate the NEWS score of the patient by summing a sub score of each piece of vital data, a sub score related to oxygen administration, and a sub score related to the presence or absence of consciousness.

In step S3, the server 2 updates the patient information table 41 illustrated in FIG. 4. In particular, the server 2 updates the patient information table 41 by updating the information indicating the vital data of each patient, the information indicating the NEWS score, the information indicating the number of hospitalization days, the information indicating the presence or absence of oxygen administration, the information indicating the presence or absence of consciousness of the patient, the information related to the medical device, and the information related to medication.

In step S4, the server 2 determines a visual mode of the patient information (vital data or the like) and the NEWS score of each of the patients A to Z, which are displayed on the patient information list screen 100. Specifically, the server 2 determines a visual mode (display color) of the vital data (respiration rate, oxygen saturation, body temperature, systolic blood pressure, and heart rate) of each of the patients A to Z, based on the sub score of the vital data. For example, in a case where the sub score of the vital data is zero points, the server 2 sets a display color of the vital data to be colorless (default display color). In a case where the sub score of the vital data is one point or two points, the server 2 sets the display color of the vital data to yellow. In a case where the sub score of the vital data is three points, the server 2 sets the display color of the vital data to red.

The display color of the vital data may be a background color of a region in which a numerical value of the vital data is displayed, or may be a character color of the numerical value of the vital data. In addition, the visual mode of the various types of information displayed on the patient information list screen 100 is not limited to the display colors of the various types of information.

On the patient information list screen 100 illustrated in FIG. 8, the respiration rate (RR) of the patient A is 10 breaths/min, and therefore, the sub score related to the respiration rate is one point. Therefore, the display color of the respiration rate of the patient A is set to yellow. The oxygen saturation (SpO2) of the patient A is 98%, and therefore, the sub score related to the oxygen saturation is zero points. Therefore, the display color of the oxygen saturation of the patient A is set to be colorless. The body temperature of the patient A is 35.9° C., and therefore, the sub score related to the body temperature is one point. Therefore, the display color of the body temperature of the patient A is set to yellow. The systolic blood pressure (Sys) of the patient A is 96 mmHg, and therefore, the sub score related to the systolic blood pressure is two points. Therefore, the display color of the systolic blood pressure of the patient A is set to yellow. The heart rate (HR) of the patient A is 35 beats/min, and therefore, the sub score related to the heart rate is three points. Therefore, the display color of the heart rate of the patient A is set to red.

In addition, in a case where oxygen is administered to the patient, the server 2 sets the display color of the information indicating the presence or absence of oxygen administration to yellow. On the other hand, in a case where oxygen is not administered to the patient, the server 2 sets the display color of the information indicating the presence or absence of oxygen administration to be colorless. In the present example, since oxygen is administered to the patient A, the display color of the information indicating the presence or absence of oxygen administration to the patient A is set to yellow.

In addition, in a case where the patient is unconscious, the server 2 sets the display color of the information indicating the presence or absence of consciousness of the patient to red. On the other hand, in a case where the patient is conscious, the server 2 sets the display color of the information indicating the presence or absence of consciousness of the patient to be colorless. In the present example, the patient A is unconscious, and therefore, the display color of the information indicating the presence or absence of consciousness of the patient A is set to red.

In addition, when there is no medical device connected to the patient, the server 2 sets the display color of the information indicating the type of the medical device to be colorless. In a case where the medical device connected to the patient is ECMO, the server 2 sets the display color of the information indicating the type of the medical device and the number of connection days to red. In a case where the medical device connected to the patient is a respirator, the server 2 sets the display color of the information indicating the type of the medical device and the number of connection days to yellow. In the present example, the ECMO is connected to the patient A, and therefore, the display color of the information indicating the type of the medical device and the number of connection days is set to red.

In addition, in a case where the number of hospitalization days of the patient is less than 20 days, the server 2 sets the display color of the information indicating the number of hospitalization days of the patient to be colorless. In a case where the number of hospitalization days of the patient is 20 days or more and 50 days or less, the server 2 sets the display color of the information indicating the number of hospitalization days of the patient to yellow. In a case where the number of hospitalization days of the patient is 51 days or more, the server 2 sets the display color of the information indicating the number of hospitalization days of the patient to red. In the present example, the number of hospitalization days of the patient A is 14 days, and therefore, the display color of the information indicating the number of hospitalization days of the patient A is set to be colorless.

In addition, in a case where there is no medication administered to the patient, the server 2 sets the display color of the information indicating the type of medication to be colorless. In a case where the medication administered to the patient is instillation, the server 2 sets the display color of the information indicating the type of medication to yellow. In a case where the medication administered to the patient is a vasopressor or an antihypertensive, the server 2 sets the display color of the information indicating the type of medication to red. In the present example, there is no medication administered to the patient A, and therefore, the display color of the information indicating the type of medication is set to be colorless. In addition, the server 2 sets the display color of the information indicating the NEWS score in accordance with a value of the NEWS score. For example, in a case where the NEWS score is less than five points, the display color of the information indicating the NEWS score may be set to be colorless. In a case where the NEWS score is five points or more and seven points or less, the display color of the information indicating the NEWS score may be set to yellow. In a case where the NEWS score is eight points or more, the display color of the information indicating the NEWS score may be set to red. In the present example, the NEWS score of the patient A is 13 points, and therefore, the display color of the information indicating the NEWS score of the patient A is set to red.

In this manner, the visual mode of the patient information and the NEWS score of each of the patients A to Z, which are displayed on the patient information list screen 100, are determined. In the description of the present embodiment, three types of display colors of red, yellow, and colorless (default display colors) are used, but the type of the display color is not particularly limited. In addition, although the display color of each piece of vital data is determined in accordance with the value of the sub score, the display color of each piece of vital data may be determined in accordance with a threshold other than the sub score. In this respect, the server 2 may set the visual mode of the information of each piece of vital data, in accordance with a comparison between the value of each piece of vital data and a predetermined threshold set for each piece of vital data.

Next, in step S5, the display terminal 3 transmits log-in information for accessing the server 2 to the server 2 via the communication network 5. Specifically, the display terminal 3 transmits the log-in information indicating a log-in ID and a password of the medical worker AA to the server 2. Authentication of the medical worker AA is not limited to log-in authentication.

In step S6, after authenticating the log-in information of the medical worker AA, the server 2 transmits, to the display terminal 3, data for displaying the patient information list screen 100 on which a plurality of pieces of patient information are displayed. Here, the data for displaying the patient information list screen 100 may be a code (HTML, CSS, JavaScript, JSON data (or XML data), or the like) for displaying the patient information list screen 100 on the WEB browser of the display terminal 3, or may be image data indicating the patient information list screen 100.

In step S7, the display terminal 3 displays the patient information list screen 100 (see FIG. 8) on the display screen of the display 34, based on the data for displaying the patient information list screen 100, which is received from the server 2. For example, the WEB browser installed in the display terminal 3 may render the patient information list screen 100 by interpreting the code (HTML, CSS, JavaScript, JSON data (or XML data), or the like) for displaying the patient information list screen 100.

According to the present embodiment, the medical worker can comprehensively determine the condition of each patient by visually recognizing the plurality of pieces of vital data displayed on the patient information list screen 100, the NEWS score, the information indicating the number of hospitalization days of the patient, and the information related to the treatment performed on the patient (that is, the information related to the medical device and the medication). In particular, the medical worker can grasp the situation of the patient (degree of intervention of the medical worker) from the viewpoint of the number of hospitalization days of the patient by recognizing the number of hospitalization days of the patient, which is an index different from the vital data and the NEWS score. In addition, the medical worker can grasp the comprehensive situation of the patient by recognizing the information related to the medical device and the medication. For example, in a case where a patient infected with a novel coronavirus is connected to the ECMO, a medical worker can accurately and comprehensively grasp a current condition of the patient, based on vital data and a NEWS score of the patient in view of the situation in which the patient is connected to the ECMO.

In addition, according to the present embodiment, the display color of the information indicating each piece of vital data is set in accordance with a comparison between the sub score of each piece of vital data and the threshold of the sub score set for each piece of vital data. In this manner, the medical worker can intuitively grasp the condition of each patient from the viewpoint of the vital data by visually recognizing the display color of the information indicating each piece of vital data displayed on the patient information list screen 100. In addition, the display color of the information indicating the NEWS score is set in accordance with a comparison between the NEWS score and the threshold set for the NEWS score. In this manner, the medical worker can intuitively grasp the condition of each patient from the viewpoint of the NEWS score by visually recognizing the display color of the information indicating the NEWS score displayed on the patient information list screen 100.

In addition, according to the present embodiment, the medical worker can intuitively grasp the situation of each patient (particularly, the degree of intervention of the medical worker) from the viewpoint of the medical device and the medication, by visually recognizing the display color of the information related to the medical device and the medication displayed on the patient information list screen 100. The medical worker can intuitively grasp the situation of each patient (particularly, the degree of intervention of the medical worker) from the viewpoint of the number of hospitalization days of the patient, by visually recognizing the display color of the information indicating the number of hospitalization days of the patient displayed on the patient information list screen 100.

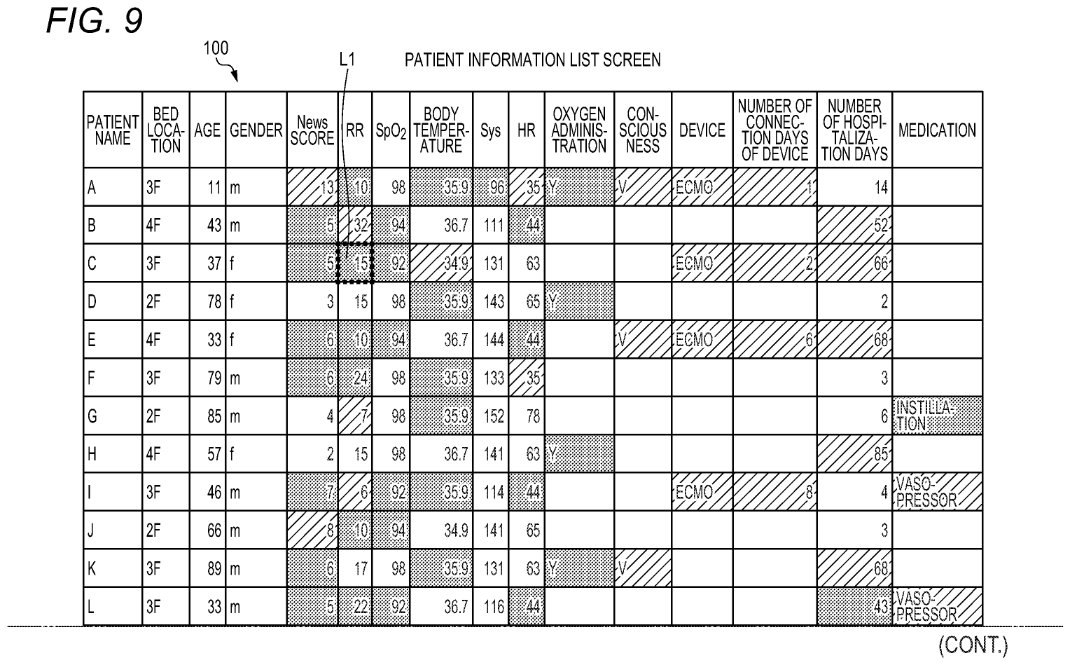

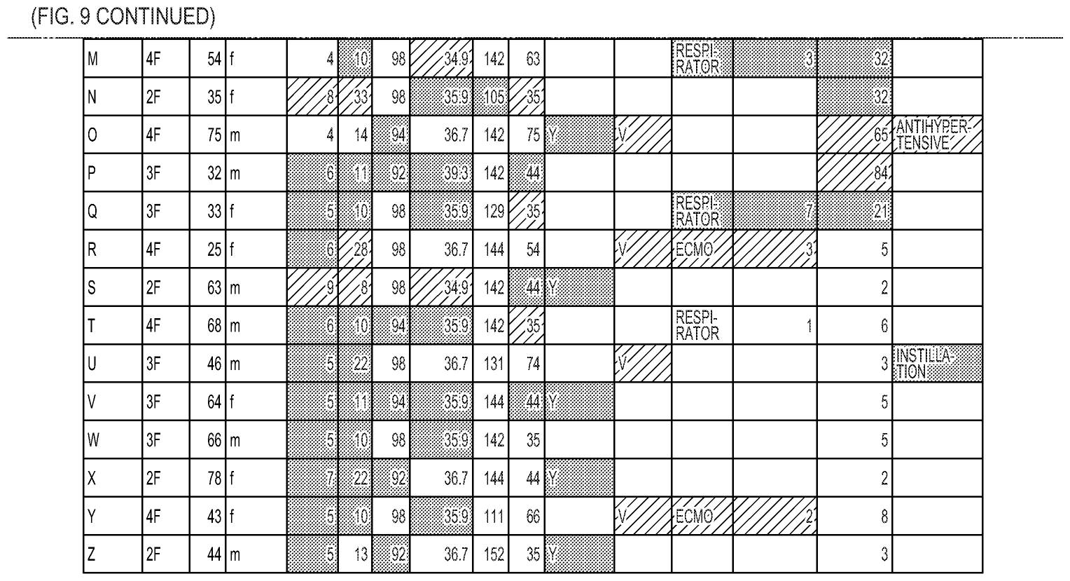

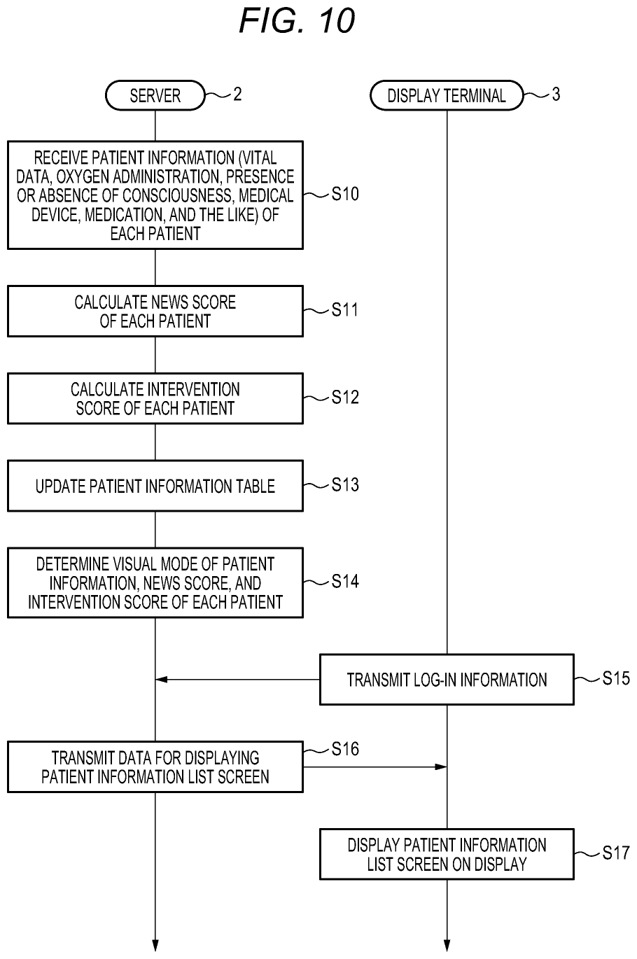

In the present embodiment, the display color of the information indicating the vital data may be changed in accordance with a specific medical device connected to the patient. In this respect, when the ECMO is worn on a patient, the display color of the information indicating the respiration rate, which is the vital data associated with wearing of the ECMO, may be changed. More specifically, in a case where the ECMO is worn on a patient, the display color of the information indicating the respiration rate may be set to yellow when the sub score related to the respiration rate is zero points or more and two points or less. In addition, in a case where the sub score related to the respiration rate is three points, the display color of the information indicating the respiration rate may be set to red. In such a case, the display color of the information indicating the respiration rate of the patient C to which the ECMO is connected is set to yellow on the patient information list screen 100 illustrated in FIG. 9 (see an area LI in FIG. 9).

That is, since the sub score related to the respiration rate of the patient Cis originally zero, the display color of the information indicating the respiration rate of the patient C is set to be colorless on the patient information list screen 100 illustrated in FIG. 8. On the other hand, since the patient C is connected to the ECMO, the display color of the information indicating the respiration rate of the patient C is changed from colorless to yellow on the patient information list screen 100 illustrated in FIG. 9. In this manner, the display color of the information indicating the respiration rate may be affected by presence or absence of wearing of the ECMO (presence or absence of a specific treatment).

In the above description, the display color of the information indicating the respiration rate of the patient wearing the ECMO is changed by changing a threshold range of the sub score related to the respiration rate. That is, in a case where the sub score related to the respiration rate is zero points or more and two points or less, the display color of the information indicating the respiration rate is set to yellow. On the other hand, the display color of the information indicating the respiration rate may be changed by changing a method of calculating the sub score related to the respiration rate.

For example, in a NEWS score calculation method illustrated in FIG. 5, the sub score related to the respiration rate is set to one point in a case where the respiration rate is nine breaths/min to 11 breaths/min. On the other hand, in a case where the ECMO is worn on the patient, the sub score related to the respiration rate may be set to one point in a case where the respiration rate of the patient is nine breaths/min to 15 breaths/min. In this case, as illustrated in FIG. 9, the respiration rate of the patient C wearing the ECMO is 15 breaths/min, and therefore, the sub score related to the respiration rate of the patient C is set to one point. Furthermore, in a case where the sub score of the vital data is one point or two points, the display color of the vital data is set to yellow, and therefore, the display color of the information indicating the respiration rate of patient C is set to yellow. In this manner, the display color of the information indicating the respiration rate may be changed by changing a reference range used for calculating the sub score related to the respiration rate.

In this manner, the display color of the information indicating the respiration rate of the patient wearing the ECMO is changed from colorless to yellow, and therefore, the medical worker can intuitively grasp the condition of the patient more suitable for reality.

An order of the processes illustrated in FIG. 7 is an example, and the order of the processes is not particularly limited. In particular, the process of step S4 may be executed not by the server 2 but by the display terminal 3. In this case, the patient information table 41 stored in the server 2 may be transmitted to the display terminal 3 before the process of step S4 is executed by the display terminal 3. In addition, the patient information list screen 100 is displayed on the display terminal 3 in the present embodiment, but the patient information list screen 100 may be displayed on the display 24 of the server 2. In this case, the medical worker AA operates the server 2 instead of the display terminal 3, whereby the patient information list screen 100 is displayed on the display 24 of the server 2.

### Second Embodiment

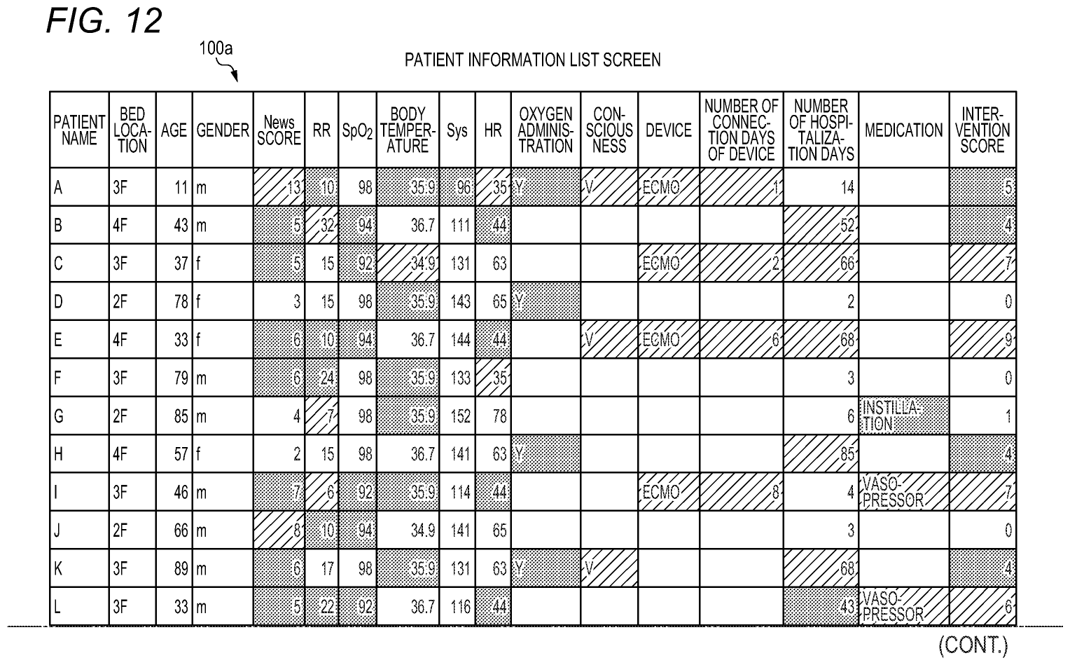

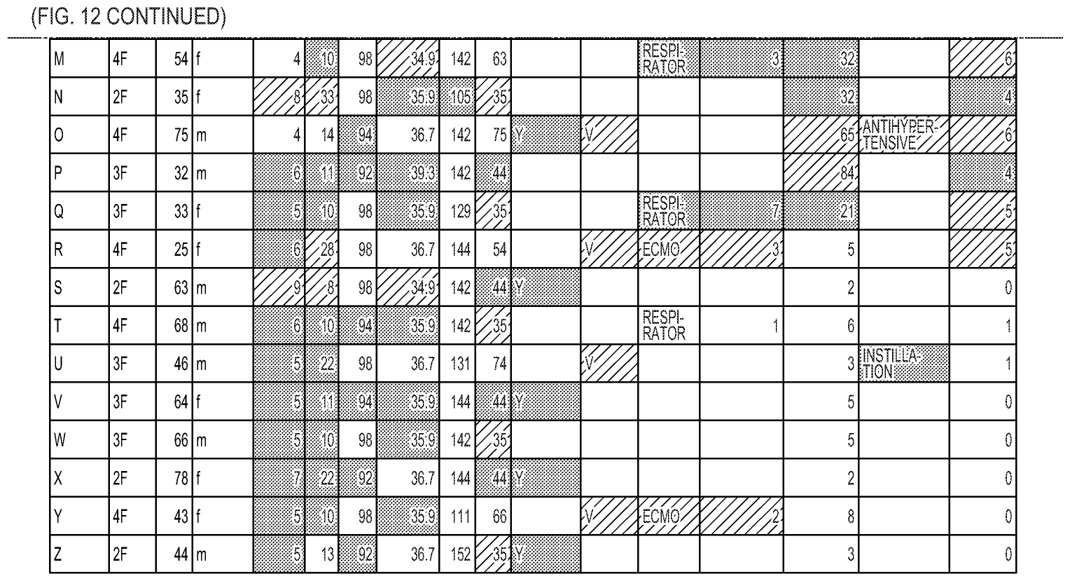

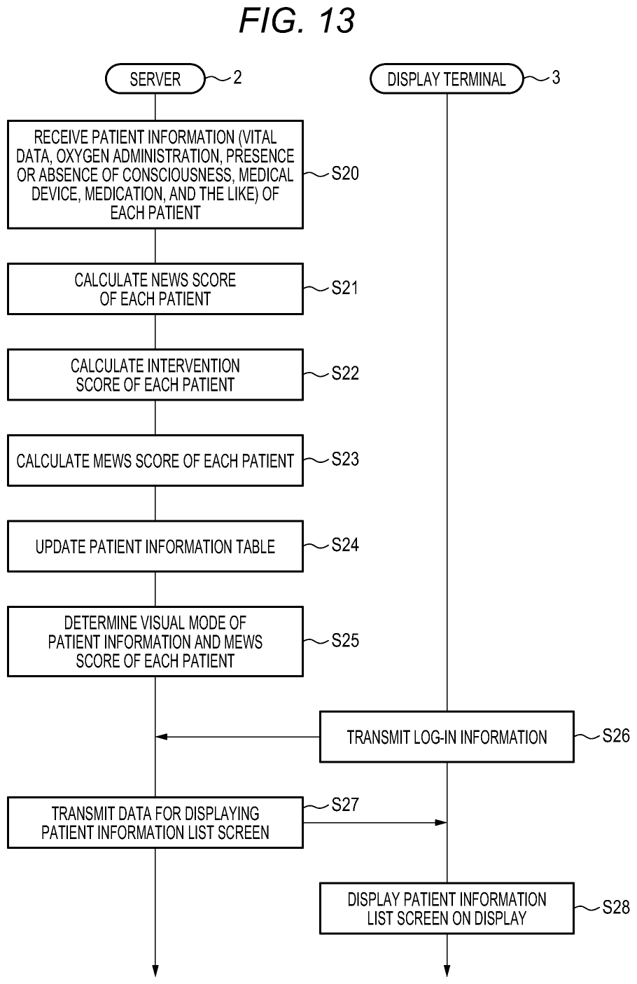

Next, a patient information processing method according to a second embodiment of the present disclosure will be described with reference to mainly FIGS. 10 and 12. FIG. 10 is a sequence diagram illustrating the patient information processing method according to the second embodiment. FIG. 11 is a diagram illustrating an example of a patient information table 41a according to the second embodiment. FIG. 12 is a diagram illustrating an example of a patient information list screen 100a according to the second embodiment. As illustrated in FIG. 10, the patient information processing method according to the second embodiment is also executed by cooperation between the server 2 and the display terminal 3.

In step S10, the server 2 receives patient information of each of the patients A to Z. In this respect, the server 2 is configured to receive a plurality of pieces of vital data (respiration rate, oxygen saturation, body temperature, systolic blood pressure, and heart rate) of each of the patients A to Z, from the plurality of bed-side monitors 6 connected to the patients A to Z via the communication network 5.

Next, in step S11, the server 2 calculates a NEWS score of each of the patients A to Z, based on a plurality of pieces of vital data of the patients A to Z, presence or absence of oxygen administration of the patients A to Z, and presence or absence of consciousness of the patients A to Z.

In step S12, the server 2 calculates an intervention score (example of intervention information) of each of the patients A to Z. Here, the intervention score is an intervention index indicating a degree of intervention of a medical worker (medical institution) with respect to a patient. The server 2 is configured to calculate the intervention score of each of the patients A to Z, based on information related to a medical device, information indicating the number of hospitalization days of the patient, and information related to medication, which are included in the patient information table 41a. More specifically, the server 2 may be configured to calculate the intervention score, by calculating each of a sub score related to the medical device, a sub score related to the number of hospitalization days of the patient, and a sub score related to medication based on a sub score table illustrated in Table 1 below, and then adding up the three sub scores.

TABLE 1
Sub Score Table

| | | | |
|---|---|---|---|
| Device | Respirator | One day to two days | 1 |
| | | Three days to four days | 2 |
| | | Five days or more | 3 |
| | ECMO | One day to two days | 3 |
| | | Three days or more | 5 |
| LOS | | Less than ten days | 0 |
| | | 11 days to 30 days | 2 |
| | | 30 days or more | 4 |
| Medication | Instillation | | 1 |
| | Vasopressor or | | 2 |
| | antihypertensive | | |

As illustrated in the sub score table, the sub score related to the medical device is determined in accordance with a type of the medical device and the number of connection days of the medical device. For example, in a case where a respirator is connected to a patient and the number of connection days of the respirator is five days or more, the sub score related to the medical device is three points. In addition, in a case where the ECMO is connected to the patient and the number of connection days of the ECMO is two days, the sub score related to the medical device is three points.

In addition, the sub score related to the number of hospitalization days of the patient is determined in accordance with the number of hospitalization days. For example, in a case where the number of hospitalization days of the patient is 15 days, the sub score related to the number of hospitalization days is two points. Furthermore, the sub score related to the medication is determined in accordance with the type of the medication. For example, in a case where an instillation is administered to a patient, the sub score related to the medication is one point.

For example, as illustrated in FIG. 11, the ECMO is connected to the patient A and the number of connection days of the ECMO is one day, and therefore, the sub score related to the medical device is three points. In addition, the number of hospitalization days of the patient A is 14 days, and therefore, the sub score related to the number of hospitalization days is two points. Medication is not administered to the patient A, and therefore, the sub score related to medication is zero points. In this manner, the intervention score of the patient A is set to five points (=3+2+0).

As described above, the server 2 is configured to calculate the intervention score of each of the patients A to Z, based on the information related to the medical device, the information indicating the number of hospitalization days of the patient, and the information related to medication by referring to the sub score table.

The sub score table illustrated in Table 1 is merely an example, and the intervention score of each patient may be calculated in accordance with another method. For example, the server 2 may be configured to calculate the intervention score of each patient, based on two pieces of information including the type of the medical device and the number of hospitalization days. In this case, the number of connection days of the medical device and the type of medication do not affect the intervention score. In addition, the server 2 may be configured to calculate the intervention score of each patient, based on two pieces of information among the type of the medical device, the type of the medication, and the number of hospitalization days of the patient. Furthermore, the server 2 may be configured to calculate the intervention score of each patient, based on any of the type of the medical device, the type of the medication, and the number of hospitalization days of the patient.

In step S13, the server 2 updates the patient information table 41a illustrated in FIG. 11. In particular, the server 2 is configured to update the patient information table 41a, by updating the information indicating the vital data of each of the patients A to Z, the information indicating the NEWS score, the information indicating the number of hospitalization days, the information indicating the presence or absence of oxygen administration, the information indicating the presence or absence of consciousness of the patient, the information related to the medical device, the information related to the medication, and the information indicating the intervention score.

In step S14, the server 2 determines a visual mode of the patient information (vital data or the like), the NEWS score, and the intervention score of each of the patients A to Z displayed on the patient information list screen 100. Specifically, the server 2 determines a visual mode (display color) of the vital data (respiration rate, oxygen saturation, body temperature, systolic blood pressure, and heart rate) of each of the patients A to Z, based on the sub score of the vital data. For example, in a case where the sub score of the vital data is zero points, the server 2 sets a display color of the vital data to be colorless (default display color). In a case where the sub score of the vital data is one or two, the server 2 sets the display color of the vital data to yellow. In a case where the sub score of the vital data is three, the server 2 sets the display color of the vital data to red.

As already described in the first embodiment, the server 2 is configured to determine the display colors of the information indicating the presence or absence of oxygen administration, the information indicating the presence or absence of consciousness of the patient, the information related to the medical device, the information indicating the number of hospitalization days of the patient, and the information indicating the type of medication. In addition, the server 2 is configured to determine the display color of the information indicating the NEWS score, in accordance with a value of the NEWS score.

Furthermore, the server 2 is configured to determine the display color of the information indicating the intervention score, in accordance with a value of the intervention score. For example, in a case where the intervention score is less than three points, the display color of the information indicating the intervention score may be set to be colorless. In a case where the intervention score is three points or more and five points or less, the display color of the information indicating the intervention score may be set to yellow. In a case where the intervention score is six points or more, the display color of the information indicating the intervention score may be set to red. In the present example, the intervention score of the patient A is five points, and therefore, the display color of the information indicating the intervention score of the patient A is set to yellow.

Next, in step S15, the display terminal 3 transmits log-in information for accessing the server 2 to the server 2 via the communication network 5. Specifically, the display terminal 3 is configured to transmit, to the server 2, the log-in information indicating a log-in ID and a password of the medical worker AA.

In step S16, after authenticating the log-in information of the medical worker AA, the server 2 transmits, to the display terminal 3, data for displaying the patient information list screen 100a on which a plurality of pieces of patient information are displayed. Here, the data for displaying the patient information list screen 100a may be a code for displaying the patient information list screen 100a on the WEB browser of the display terminal 3, or may be image data indicating the patient information list screen 100a.

In step S17, the display terminal 3 displays the patient information list screen 100a (see FIG. 12) on a display screen of the display 34, based on the data for displaying the patient information list screen 100a, which is received from the server 2. The patient information list screen 100a illustrated in FIG. 12 is different from the patient information list screen 100 illustrated in FIG. 8 in that information indicating the intervention score of the patient is displayed.

According to the present embodiment, the medical worker can comprehensively determine the condition of each patient, by visually recognizing the plurality of pieces of vital data, the NEWS score, and the intervention score, which are displayed on the patient information list screen 100a. In particular, the medical worker can grasp the degree of intervention of the medical worker by recognizing the intervention score. In this manner, the medical worker can determine a comprehensive condition of each patient based on the degree of intervention of the medical worker.

In addition, according to the present embodiment, the display colors of the patient information, the NEWS score, and the intervention score of each patient are set, in accordance with respective predetermined thresholds, and therefore, the medical worker can intuitively grasp the condition of each patient by visually recognizing the display color of the information displayed on the patient information list screen 100a.

### Third Embodiment

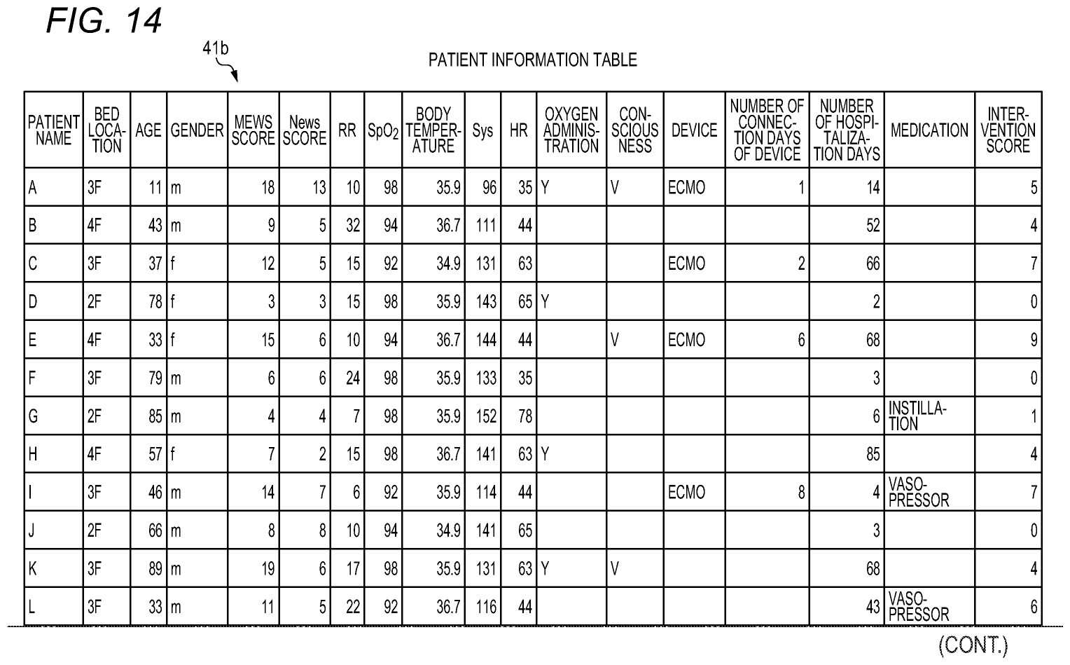

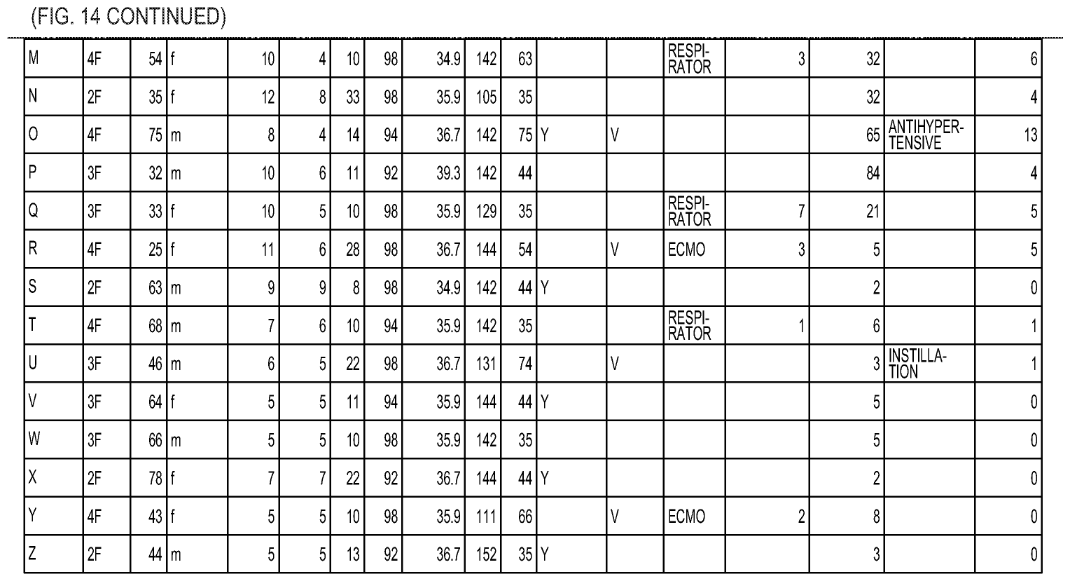

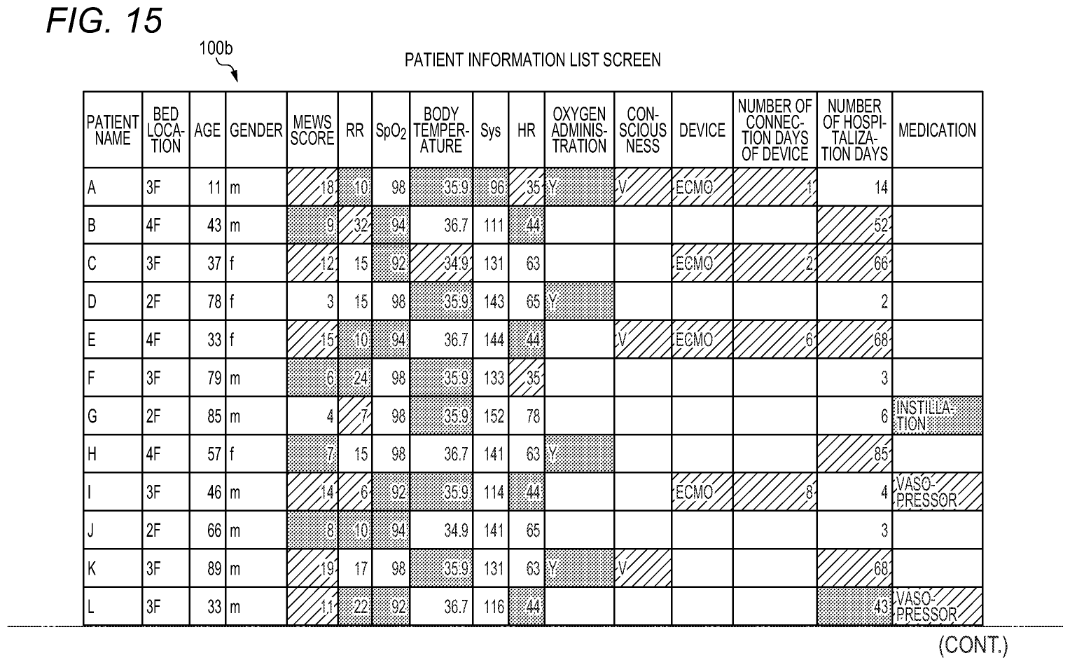

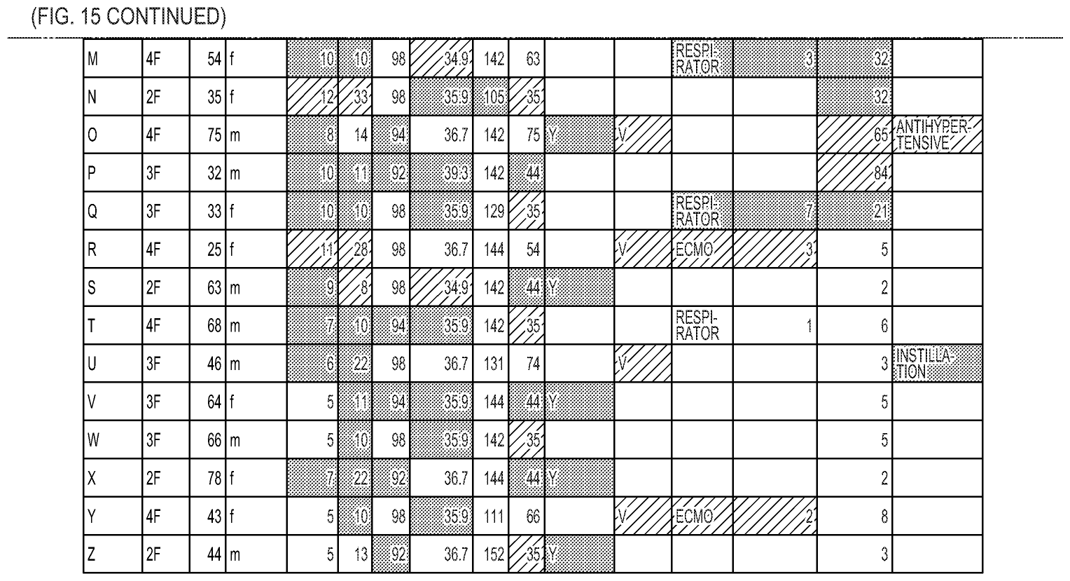

Next, a patient information processing method according to a third embodiment of the present disclosure will be described with reference to mainly FIGS. 13 and 15. FIG. 13 is a sequence diagram illustrating the patient information processing method according to the third embodiment. FIG. 14 is a diagram illustrating an example of a patient information table 41b according to the third embodiment. FIG. 15 is a diagram illustrating an example of a patient information list screen 100b according to the third embodiment. As illustrated in FIG. 13, the patient information processing method according to the third embodiment is also executed by cooperation between the server 2 and the display terminal 3.

In step S20, the server 2 receives the patient information of each of the patients A to Z. In this respect, the server 2 is configured to receive a plurality of pieces of vital data (respiration rate, oxygen saturation, body temperature, systolic blood pressure, and heart rate) of each of the patients A to Z, from the plurality of bed-side monitors 6 connected to the patients A to Z via the communication network 5.

Next, in step S21, the server 2 calculates a NEWS score of each of the patients A to Z, based on a plurality of pieces of vital data of the patients A to Z, presence or absence of oxygen administration of the patients A to Z, and presence or absence of consciousness of the patients A to Z.

In step S22, the server 2 calculates an intervention score of each of the patients A to Z. The server 2 is configured to calculate the intervention score of each of the patients A to Z, based on information related to a medical device, information indicating the number of hospitalization days of the patient, and information related to medication, which are included in the patient information table 41b. Since a specific method of calculating the intervention score has already been described in the second embodiment, the description thereof is omitted here.

In step S23, the server 2 calculates a modified NEWS (MEWS) score of each of the patients A to Z. Here, the MEWS score (example of the vital score) is an index calculated based on a NEWS score and an intervention score. More specifically, the server 2 is configured to calculate the MEWS score by summing up the NEWS score and the intervention score. That is, the MEWS score is also a total value of each of sub scores configuring the NEWS score (that is, the sub score of each piece of vital data) and each of sub scores configuring the intervention score (that is, a sub score related to a medical device, a sub score related to the number of hospitalization days of a patient, and a sub score related to medication). In this manner, the MEWS score includes a NEWS score component indicating a condition of the patient and an intervention score component indicating a degree of intervention of the medical worker, and thus may be used as an index comprehensively indicating the condition of the patient.

For example, as illustrated in FIG. 14, the NEWS score of the patient A is 13 points and the intervention score of the patient A is five points, and therefore, the MEWS score of the patient A is 18 points (=13+5).

In step S24, the server 2 updates the patient information table 41b illustrated in FIG. 14. In particular, the server 2 is configured to update the patient information table 41b, by updating information indicating vital data of each of the patients A to Z, information indicating the NEWS score, information indicating the number of hospitalization days, information indicating presence or absence of oxygen administration, information indicating presence or absence of consciousness of the patient, information related to a medical device, information related to medication, information indicating the intervention score, and information indicating the MEWS score.

In step S25, the server 2 determines a visual mode of the patient information (vital data or the like) and the MEWS score of each of the patients A to Z, which are displayed on the patient information list screen 100b. As already described in the first embodiment, the server 2 is configured to determine the display colors of the information indicating each piece of vital data, the information indicating the presence or absence of oxygen administration, the information indicating the presence or absence of consciousness of the patient, the information related to the medical device, the information indicating the number of hospitalization days of the patient, and the information indicating the type of medication.

The server 2 is configured to determine the display color of the information indicating the MEWS score in accordance with a value of the MEWS score. For example, in a case where the MEWS score is less than five points, the display color of the information indicating the MEWS score may be set to be colorless (default display color). In a case where the MEWS score is six points or more and ten points or less, the display color of the information indicating the MEWS score may be set to yellow. In a case where the MEWS score is 11 points or more, the display color of the information indicating the MEWS score may be set to red. In the present example, the MEWS score of the patient A is 18 points, and therefore, the display color of the information indicating the MEWS score of the patient A is set to red.

Next, in step S26, the display terminal 3 transmits log-in information for accessing the server 2 to the server 2 via the communication network 5. Specifically, the display terminal 3 is configured to transmit, to the server 2, the log-in information indicating a log-in ID and a password of the medical worker AA.

In step S27, after authenticating the log-in information of the medical worker AA, the server 2 transmits, to the display terminal 3, data for displaying the patient information list screen 100b on which a plurality of pieces of patient information are displayed. Here, the data for displaying the patient information list screen 100b may be a code for displaying the patient information list screen 100b on the WEB browser of the display terminal 3, or may be image data indicating the patient information list screen 100b.

In step S28, the display terminal 3 displays the patient information list screen 100b (see FIG. 15) on a display screen of the display 34, based on the data for displaying the patient information list screen 100b, which is received from the server 2. The patient information list screen 100b illustrated in FIG. 15 is different, from the patient information list screen 100 illustrated in FIG. 8 and the patient information list screen 100a illustrated in FIG. 12, in that information indicating the MEWS score of the patient is displayed.

According to the present embodiment, the medical worker can comprehensively determine a condition of each patient by visually recognizing the plurality of pieces of vital data and the MEWS score. In particular, by recognizing the MEWS score, the medical worker can determine the comprehensive condition of each patient, based on the degree of intervention of the medical worker.

In addition, in order to implement the patient information processing method according to the present embodiment by software, a patient information processing program may be incorporated, in advance, in a storage device and/or the ROM of the server 2 and/or the display terminal 3. Alternatively, the patient information processing program may be stored in a non-transitory computer readable storage medium such as a magnetic disk (for example, HDD and a floppy disk), an optical disk (for example, CD-ROM, DVD-ROM, and Blu-ray (registered trademark) disk), a magneto optical disk (for example, MO), or a flash memory (for example, an SD card, a USB memory, and SSD). In this case, the patient information processing program stored in the storage medium may be incorporated in the storage device. Further, after the program incorporated in the storage device is loaded onto RAM, one or more processors may execute the program loaded on RAM. In this manner, the patient information processing method according to the present embodiment is executed by the server 2 and/or the display terminal 3. In addition, the patient information processing program may be downloaded from an external computer on a communication network. In this case, the downloaded program may be also incorporated in the storage device.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A patient information processing method executed by one or more processors, the patient information processing method comprising:
a step of obtaining a plurality of pieces of vital data of a patient;
a step of obtaining information indicating a content and a time of a treatment performed on the patient; and
a step of causing a display to display information indicating at least one of the plurality of pieces of vital data or an index associated with the plurality of pieces of vital data, and to display information indicating the content and the time of the treatment.

2. The patient information processing method according to claim 1, further comprising:
a step of setting a visual mode of the information indicating the content and the time of the treatment, in accordance with the content and/or the time of the treatment.

3. The patient information processing method according to claim 1, further comprising:
a step of setting a visual mode of the information indicating the plurality of pieces of vital data, in accordance with a comparison between a value of each of the plurality of pieces of vital data and a threshold set for each of the plurality of pieces of vital data.

4. The patient information processing method according to claim 1, further comprising:
a step of obtaining information indicating a hospitalization time of the patient; and
a step of causing the display to further display the information indicating the hospitalization time of the patient.

5. The patient information processing method according to claim 4, further comprising:
a step of setting a visual mode of the information indicating the hospitalization time of the patient, in accordance with a comparison between the hospitalization time and a threshold set for the hospitalization time.

6. The patient information processing method according to claim 3,
wherein the plurality of pieces of vital data includes first vital data related to a specific treatment, and
the step of setting a visual mode of information indicating the first vital data includes:
a step of changing a first threshold set for the first vital data, in a case where the specific treatment is performed on the patient; and
a step of setting a visual mode of the information indicating the first vital data, in accordance with a comparison between a value of the first vital data and the changed first threshold.

7. The patient information processing method according to claim 1, further comprising:
a step of calculating a vital score indicating a condition of the patient, at least based on the plurality of pieces of vital data,
wherein the index associated with the plurality of pieces of vital data is the vital score.

8. The patient information processing method according to claim 7,
wherein the step of calculating a vital score includes:
a step of calculating a sub score of each of the plurality of pieces of vital data, in accordance with a comparison between each of the pieces of plurality of vital data and a reference range set for each of the plurality of pieces of vital data; and
a step of calculating the vital score, based on each calculated sub score, the plurality of pieces of vital data include first vital data related to a specific treatment, and
the step of calculating a first sub score of the first vital data includes:
a step of changing a first reference range set for the first vital data, in a case where the specific treatment is performed on the patient; and
a step of calculating the first sub score, in accordance with a comparison between a value of the first vital data and the changed first reference range.

9. The patient information processing method according to claim 7,
wherein the step of calculating a vital score includes:
a step of calculating a sub score of each of the plurality of pieces of vital data, based on a comparison between each of the plurality of pieces of vital data and a reference range set for each of the plurality of pieces of vital data;
a step of calculating a sub score related to a treatment performed on the patient, based on information related to the treatment; and
a step of calculating the vital score, at least based on the sub score of each of the plurality of pieces of vital data and the sub score related to the treatment.

10. The patient information processing method according to claim 9,
wherein the step of calculating a vital score includes:
a step of calculating a sub score related to the hospitalization time, based on information indicating a hospitalization time of the patient; and
a step of calculating the vital score, based on a sub score of each of the plurality of pieces of vital data, a sub score related to the treatment, and a sub score related to the hospitalization time.

11. The patient information processing method according to claim 1,
wherein the information indicating the content and the time of the treatment performed on the patient includes information indicating a type and a connection time of a medical device connected to the patient.

12. A patient information processing method executed by one or more processors, the patient information processing method comprising:
a step of obtaining a plurality of pieces of vital data of a patient;
a step of calculating intervention information indicating a degree of intervention of a medical worker with respect to the patient, based on at least one of information related to a treatment performed on the patient or information indicating a hospitalization time of the patient; and
a step of causing a display to display the intervention information and information indicating at least one of the plurality of pieces of vital data or an index associated with the plurality of pieces of vital data.

13. The patient information processing method according to claim 12,
wherein the information related to the treatment includes at least one of information indicating a content of the treatment or information indicating a time of the treatment.

14. The patient information processing method according to claim 13, wherein the information related to the treatment includes information indicating a type of a medical device connected to the patient.

15. A non-transitory computer readable storage medium storing a program comprising instructions which, when executed by a computer, cause the computer to execute the patient information processing method according to claim 1.

16. A non-transitory computer readable storage medium storing a program comprising instructions which, when executed by a computer, cause the computer to execute the patient information processing method according to claim 12.

17. A patient information processing device comprising:
one or more processors; and
one or more memories configured to store a computer-readable instruction,
wherein, in a case where the computer-readable instruction is executed by the one or more processors, the patient information processing device is configured to:
obtain a plurality of pieces of vital data of a patient;
obtain information indicating a content and a time of a treatment performed on the patient; and
output information indicating at least one of the plurality of pieces of vital data or an index associated with the plurality of pieces of vital data, and information indicating the content and the time of the treatment.

18. A patient information processing device comprising:
one or more processors; and
one or more memories configured to store a computer-readable instruction,
wherein, in a case where the computer-readable instruction is executed by the one or more processors, the patient information processing device is configured to:
obtain a plurality of pieces of vital data of a patient;
calculate intervention information indicating a degree of intervention of a medical worker with respect to the patient, based on at least one of information related to a treatment performed on the patient or information indicating a hospitalization time of the patient; and
output the intervention information and information indicating at least one of the plurality of pieces of vital data or an index associated with the plurality of pieces of vital data.

19. A patient information processing system comprising:
a server; and
a display terminal communicably connected to the server,
wherein the patient information processing system is configured to:
obtain a plurality of pieces of vital data of a patient;
obtain information indicating a content and a time of a treatment performed on the patient; and
cause a display to display information indicating at least one of the plurality of pieces of vital data or an index associated with the plurality of pieces of vital data, and information indicating the content and the time of the treatment.

20. A patient information processing system comprising:
a server; and
a display terminal communicably connected to the server,
wherein the patient information processing system is configured to:
obtain a plurality of pieces of vital data of a patient;
calculate intervention information indicating a degree of intervention of a medical worker with respect to the patient, based on at least one of information related to a treatment performed on the patient or information indicating a hospitalization time of the patient; and
cause a display to display the intervention information and information indicating at least one of the plurality of pieces of vital data or an index associated with the plurality of pieces of vital data.
